(19) United States
(12) Patent Application Publication
(10) Pub. No.: US 2011/0218410 A1
(43) Pub. Date: Sep. 8, 2011

(54) REMOTE PATIENT MANAGEMENT SYSTEM

(75) Inventors: Harm Jacob BUISMAN, EINDHOVEN (NL); Aleksandra TESANOVIC, EINDHOVEN (NL)

(73) Assignee: KONINKLIJKE PHILIPS ELECTRONICS N.V., EINDHOVEN (NL)

(21) Appl. No.: 13/027,537

(22) Filed: Feb. 15, 2011

Related U.S. Application Data
(60) Provisional application No. 61/310,065, filed on Mar. 3, 2010.

(51) Int. Cl. A61B 5/00; G06Q 50/00; G06Q 10/00

(52) U.S. Cl. 600/301; 705/3; 600/300

(57) ABSTRACT

The remote patient management system comprises: a computing device with a processor, a home infrastructure device comprising a patient display, a healthcare professional display, and a computer readable storage medium. The computer readable storage medium contains instructions that cause the processor to perform a method of displaying images on the patient display during a teleconsultation. The method comprises the steps of: accessing a patient record from a patient record database, creating a patient profile using the patient record, identifying a consultation aim using the patient profile, determining a list of topics using the consultation aim, creating a teleconsultation outline using the list of topics, selecting a set of images using the list of topics, displaying the teleconsultation outline on the healthcare professional display during the teleconsultation, and displaying the images on the patient display during the teleconsultation.

### TECHNICAL FIELD

The invention relates to remote patient management systems, in particular remote patient management systems adapted for displaying images on a patient display.

### BACKGROUND OF THE INVENTION

A large part of patient-medical professional communication, for example face-to-face consults and phone interactions, is spent on information exchange. This is particularly true for educating the patient about his or her medical condition, current health situation, treatment options, and consequences of non-compliant behavior. The reason this is done is so that the patient adheres to the medical interventions or care plan. For guaranteeing effectiveness of medical interventions is it essential that the patient understands the information provided by the medical or healthcare professionals.

Currently, information is provided to the patient during the interaction with the professional by explaining them orally about a certain topic. Sometimes (only during face-to-face consults) using a printed image that shows in general terms the condition. Over the phone this is done using only voice communication without any visual aids.

Research and practice show that the voice communication is not optimal for retention and understanding of the topics that are being discussed: Retention rates by only voice communication are low, and normally range up to 20%. This implies that of all information provided to the patient by health professional not even quarter of it is remembered by the patient. On the other side, the research shows that retention rates in general communication depend heavily on communication mode used. We see that by reading only, one can retain on average 10%, while viewing/seeing only is around 30%, while combination of hearing and reading improves retention up to 50%.

### SUMMARY OF THE INVENTION

Embodiments of the invention may increase the effectiveness of a teleconsultation between a patient and a healthcare professional a method of displaying images on a patient display during a teleconsultation is performed by a remote patient monitoring system according to an embodiment of the invention. The effectiveness of a teleconsultation can either be a measure of the amount of material from a teleconsultation that is remembered by the patient or a change in the behavior of the patient. For instance a quiz could be used to test patient knowledge after the teleconsultation or the patient's health and/or behavior after the teleconsultation could be monitored. Additionally, embodiments of the invention may increase the compliance to treatment, due to more knowledgeable patients, and increased engagement of the patients.

Embodiments of the invention may have the following features:

A display on the patient's end to display visual content

A display on the professional end, that allows for pre-viewing and selecting visual content to be shown at the patient's end in real time

A storage to store visual content

A communications means to allow for voice communication, and transmission of the content

The visual content is automatically presented as a nurse goes through a call script (e.g. workflow)

There is a means of highlighting specific elements in the visual content, by the professional and/or by the patient

The visual content presented to the professional that can be shown to the patient is selected/suggested by an algorithm based on the relevance of this content

The algorithm is self-learning based on historic use

The workflow/call script is automatically selected based on the type of alert or type of planned topic of teleconsult

Difficulties or problems which embodiments of the invention may solve are:

High health professional workload: Communication efficiency during the consultation. Currently, for example, a nurse spends on average 8 minutes to call a patient of which most of the time is spent on patient education on their disease and treatment. The communication means used is only voice communication, where the nurse first assesses what is patient's current health situation and then provides voice counseling. This leads to low retention of the information given by the nurse, resulting in an overhead in repeated explanation during the following consult or patient non-compliance leading to worsening of the condition and often re-hospitalizations.

Low patient compliance, due to generic information/education: Delivery of patient-tailored education during patient-professional encounter (especially phone counseling). Tailored education (including communication) may plays an important role for addressing non-compliance. As mentioned earlier non-compliance is the most commonly identified cause of disease worsening, e.g., heart failure (HF) decomposition. Non-compliance is the precipitating factor of exacerbation, leading to poor clinical outcomes. Tailoring of delivered content today is done by the nurse first discussing with the patient what is his or her current situation and then discussing the key topics that are most relevant to this patient. This leads to reduced communication efficiency (discussed in previous bullet), and also worsened non-compliance of the patient.

Low patient information retention during teleconsultations: Improving retention of the education material, which directly impacts the first two named problems.

A ‘computing device’ as used herein refers to any device comprising a processor. A processor is an electronic component which is able to execute a program or machine executable instruction. References to the computing device comprising “a processor” should be interpreted as possibly containing more than one processor. The term computing device should also be interpreted to possibly refer to a collection or network of computing devices each comprising a processor. Many programs have their instructions performed by multiple processors that may be within the same computing device or which may even distributed across multiple computing device.

A ‘computer readable storage medium’ as used herein is any storage medium which may store instructions which are executable by a processor of a computing device. In some embodiments, a computer readable storage medium may also be able to store data which is able to be accessed by the processor of the computing device. An example of a computer readable storage medium include, but are not limited to: a floppy disk, a magnetic hard disk drive, a solid state hard disk, flash memory, a USB thumb drive, Random Access Memory (RAM) memory, Read Only Memory (ROM) memory, an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. The term computer readable storage medium also refers to various types of recording media capable of being accessed by the computer device via a network or communication link. For example a data may be retrieved over a modem, over the internet, or over a local area network.

‘Computer memory’ as used herein is an example of a computer readable storage medium. Computer memory is any memory which is directly accessible to a processor. Examples of computer memory include, but are not limited to: RAM memory, registers, and register files.

‘Computer storage’ as used herein is an example of a computer readable storage medium. Computer storage is any non-volatile computer readable storage medium. Examples of computer storage include, but are not limited to: a hard disk drive, a USB thumb drive, a floppy drive, a smart card, a DVD, a CD-ROM, and a solid state hard drive. In some embodiments computer storage may also be computer memory or vice versa.

A ‘display’ as used herein is an electronic device adapted for graphically displaying text, images, multimedia clips, video, and other visual content. Examples of a display include, but are not limited to: a computer monitor, the screen of a cellular telephone, and a television. A ‘patient display’ as used herein is a display. A ‘healthcare professional display’ as used herein is a display.

A ‘remote patient management system’ as used herein is a system for remotely administering a care plan. A care plan is a day-to-day plan for managing a disease or health condition.

A ‘content element’ as used herein is content which may be provided to a patient and which may be integrated into a care plan for the patient. For instance a remote patient management system may present content elements for education or motivating a patient.

A ‘care plan’ is a day-to-day plan for managing a disease or health condition. Content elements may be provided either by a hospital or an outpatient clinic or a disease management organization or a remote patient management system.

A ‘home infrastructure device’ as used herein is a device adapted for delivering the content element and the assessment content element to the patient. In some embodiments, a home infrastructure device may comprise at least one diagnostic medical device for measuring a value of a patient's vital sign.

The term ‘vital sign’ as used herein refers to are any physical property of the patient which may be measured. Examples of vital signs include, but are not limited to: weight, blood sugar level, blood pressure, pulse, SpO2, and bio-impedance

A ‘teleconsultation’ as used herein consultation between a healthcare professional and a patient that is conducted using telecommunications or communications equipment. A teleconsultation may be performed remotely via telecommunications. For example, a teleconsultation may be conducted via, but not limited to: telephone, radio, voice of internet protocol, teleconferencing equipment, cellular telephone, or video calling, other telecommunications medium. Teleconsultation is typically performed with the healthcare professional and the patient being at separate locations. In this case the telecommunications equipment is needed for the healthcare professional and the patient to communicate. However, a teleconsultation may also be performed if the patient and the physician are within audible distance from each other. In this case the telecommunications equipment is not used for the communication between the patient and the physician, but is instead used to record the speech of the patient and the physician during the teleconsultation.

A ‘teleconsultation outline’ as used herein is an outline or transcript which is prepared for a healthcare professional to use during a teleconsultation. A ‘teleconsultation outline’ may be an outline or graphically displayed nodes which outline the predetermined contents of a teleconsultation.

A ‘health professional interface’ as used herein is a user interface for a computing device which allows a health professional to control and/or interact with a remote patient management system.

A ‘patient interface’ as used herein is a user interface for a computing device which allows a patient to interact a remote patient management system and/or receive a content element from a remote patient management system.

The invention provides for a remote patient management system. The remote patient management system comprises a computing device comprising a processor. The remote patient management system further comprises a home infrastructure device comprising a patient display. The remote patient management system further comprises a healthcare professional display. The remote patient management system further comprises a computer readable storage medium. The computer readable storage medium contains instructions that when executed by the processor cause the processor to perform a method of displaying images on the patient display during a teleconsultation.

The method comprises the step of accessing a patient record from a patient record database. A patient record as used herein is a record of a particular patient's descriptive data, medical history and other health records. For instance a patient record may contain a treatment or care plan. A patient record database as used herein is a file or database which contains a collection of patient records. The patient record database may for instance be a file or collection of files stored on a server. The patient record database may also be a so called electronic collection of electronic health records. In this step a patient record belonging to a particular patient is accessed or retrieved.

The method further comprises the step of creating a patient profile using the patient record. In this step the data which is contained in the patient record is examined and a patient profile is created. The patient profile may be created by examining the data and determining if the patient has data which indicates the need for treatment or counseling on a particular medical condition or topic. For instance the patient profile may have a record of the patient being diagnosed with diabetes. In this case the patient record could be examined for data which indicates that the patient's diabetes is not being properly managed. The patient profile could also be examined for recent changes in a patient's diagnosis. For instance if a patient was recently diagnosed with heart disease this could be put into the patient profile and the patient could be profiled as a patient with recently diagnosed heart disease. This could be beneficial because the patient having this new condition may need counseling on how to properly manage his or her medical condition. The method further comprises the step of identifying a consultation aim using the patient profile. The consultation aim as used herein is a goal or a list of deliverables to be achieved during a teleconsultation. For instance in the case of the patient recently diagnosed with heart disease the consultation aim could be to inform the patient of his or her condition. For the previously mentioned diabetic the consultation aim may be to better manage one's diet so as to control blood sugar levels.

The method further comprises the step of determining a list of topics using the consultation aim. Using the consultation aim a list of topics can be constructed to meet the consultation aim. For instance in the case of the patient with recently diagnosed heart disease these could be a list of topics which the healthcare professional should discuss with the patient in order to inform him or her of the condition. For the case of the diabetic the list of topics may include specific information or types of training that the healthcare professional can engage in with the patient during the teleconsultation.

The method further comprises the step of creating a teleconsultation outline using the list of topics. A teleconsultation outline as used herein is an outline or script which may be used by a healthcare professional during a teleconsultation. The teleconsultation outline may have a variety of forms. Teleconsultation outlines may be displayed in a fashion similar to a teleprompter. The teleconsultation outline may also be displayed in an outline form so that the healthcare professional may see the flow of the conversation.

The method further comprises the step of collecting a set of images using the list of topics. For each of the topics a set of images may be selected. The images may be selected from a database or there may be predetermined images which correspond to a particular topic or topics. The method further comprises the step of displaying the teleconsultation outline on the healthcare professional display during the teleconsultation. The method further comprises the step of displaying images on the patient display during the teleconsultation. This is advantageous because during the teleconsultation the teleconsultation outline is displayed on a healthcare professional display and this is able to guide and remind the healthcare professional which topics to discuss and provide information about during the teleconsultation. During the same time images on the patient display are displayed during the teleconsultation. This is advantageous because the images work as a graphical reinforcement to the teleconsultation by the healthcare professional.

In another embodiment the home infrastructure device comprises at least one diagnostic medical device for measuring a value of a first patient vital sign. The method of displaying images on the patient display during a teleconsultation is initiated when the value of the first patient vital sign is outside of a predetermined range. This embodiment is advantageous because the home infrastructure device can use the at least one diagnostic medical device to periodically or repeatedly monitor the value of the first patient vital sign. When the first patient vital sign goes outside of the predetermined range then this may indicate that the patient has a health condition which needs to be addressed by a healthcare professional or that the patient is not managing his or her medical condition properly. In this case the intervention of a healthcare professional is needed. The remote patient management system initiates the method automatically and prepares the teleconsultation outline for the healthcare provider to use with the teleconsultation. This is extremely beneficial, because it reduces the chances that a patient who has a change in his or her health condition will not receive timely medical care. For instance if a patient is monitoring his or her own blood sugar level, if the blood sugar level becomes too high the patient may or may not call a physician to discuss the problem. If the device for measuring the blood sugar level is integrated into the home infrastructure device if there is a problem with the blood sugar level the method will be activated automatically and the patient will receive a teleconsultation with a healthcare professional that can address the patient's health issues.

In another embodiment the home infrastructure device comprises at least one diagnostic medical device for measuring the value of a second patient vital sign. The determination of the list of topics is at least partially performed using the value of the second patient vital sign. In this embodiment the value of the second patient vital sign and the patient record are used to identify the consultation aim. Essentially the patient's medical history and the patient's current medical condition as according to the home infrastructure device are used to identify the consultation aim. This embodiment is advantageous because the teleconsultation outline and images selected will be appropriate for the patient's medical history and also the current medical condition of the patient. The first patient vital sign and the second patient vital sign may be different vital signs or they may be the same vital sign.

In another embodiment there is a portion of the teleconsultation outline that corresponds to each image of the set of images. The healthcare professional display is adapted for emphasizing the portion of the teleconsultation outline. The patient display is adapted for displaying the corresponding image when the portion is emphasized. This embodiment is advantageous because the topics or some of the topics of the teleconsultation outline may be displayed on the healthcare professional display. In one version of this embodiment the text is emphasized sequentially so that the healthcare professional knows which topic is being discussed.

In another embodiment the healthcare professional is able to see multiple items of the teleconsultation outline and select which is to be emphasized. When the item for the emphasis is selected then the image corresponding to the selected element of the outline is displayed on the patient display.

In another embodiment the healthcare professional display comprises a healthcare professional user interface. The method further comprises displaying a list of topics on the healthcare professional's user interface wherein the method further comprises receiving edit data for the list of topics. The method further comprises editing the list of topics using the edit data. This embodiment is advantageous because after the list of topics is determined it is displayed so that the healthcare professional may edit the list of topics. Once the list of topics is edited then the teleconsultation outline and the images are selected automatically. In this embodiment more feedback from the healthcare professional is allowed for.

In another embodiment the home infrastructure device comprises at least one diagnostic medical device for measuring a value of a third patient vital sign. The method further comprises instructing the home infrastructure device to record a log of the value of the third patient vital sign for a predetermined amount of time. The determination of the list of topics is at least partially performed using the log. In this embodiment the diagnostic medical device measures the value of the third patient vital sign for a predetermined amount of time. During this time it is logged. This is beneficial because it allows the use of the home infrastructure device to track how the value of the third patient vital sign is changing through time which allows an identification of health problems which are changing with the patient. For instance the patient may gradually gain weight over time. By analyzing this and including it in the list of topics the healthcare professional can address behavior by the patient which is changing or worsening over time. The third patient vital sign and the first patient vital sign may be different vital signs or they may be the same vital sign. The third patient vital sign and the second patient vital sign may be different vital signs or they may be the same vital sign.

In another embodiment the patient profile is created by collecting information which indicates a health risk. The patient profile may be examined for information which indicates a health risk. This may be achieved by using pattern recognition. There may also be the record of vital signs within the health record. If these vital signs are outside of a predetermined range then this may indicate a health risk. Previous illnesses and/or diagnoses may also indicate that the patient has a health risk, for instance if the patient is currently being treated for alcoholism, a previous diagnosis of diabetes may indicate a health risk.

In another embodiment the patient display comprises a patient user interface adapted for receiving input from a patient. The patient user interface is adapted for receiving a selection of a displayed image. The displayed image is one of the set of images that were selected using the list of topics. The home infrastructure device is adapted for sending a signal to the computing device which indicates the selection of the displayed image using the patient user interface. The method further comprises displaying predetermined educational content on the patient display when the signal is received. In this embodiment the patient is able to select an image which is displayed on the patient display. When the image is selected educational content could be provided to the patient. This may take several different forms, for instance when an image is selected a predetermined content element may be delivered to the home infrastructure device. This may occur during the consultation or it may occur after the consultation.

In another embodiment the patient user interface comprises a microphone. The patient user interface is adapted for receiving voice commands using the microphone for receiving the selection of a displayed image. This embodiment is advantageous because a patient may be disabled and the use of a voice command to make the selection of a displayed image may make use of the patient management system easier for the patient.

In another embodiment the method further comprises selecting educational content from an educational content database using the list of topics. The method further comprises delivering the educational content to the home infrastructure device for display on the patient display. The method further comprises the step of presenting the educational content using the patient display. The educational content may be considered to be a content element. The educational content may be displayed during the teleconsultation. Alternatively the educational content may be displayed a predetermined day and time after the teleconsultation. In this way the remote patient management system may be used to reinforce the teleconsultation. For instance after the teleconsultation, a video may be shown which reinforces the discussion that the patient had during the teleconsultation.

In another embodiment the method further comprises constructing a quiz from a quiz database using the list of topics. The quiz database may be a file containing questions or a database containing a selection of questions and answers. There may be one or more questions corresponding to each of the list of topics. The method further comprises delivering the quiz to the home infrastructure device for display on the patient display. In this respect the quiz may be better to be a content element. The method further comprises the step of presenting the quiz using the patient display. The method further comprises the step of scoring the quiz. The method further comprises the step of evaluating the effectiveness of the teleconsultation at least partially using the scored quiz. The use of a quiz in such a way may be beneficial because it may help reinforce the knowledge of the patient gained during the teleconsultation. The quiz may serve to remind the patient of the topics that were discussed, or the knowledge that a quiz will be given may encourage the patient to pay more attention during the teleconsultation.

In another embodiment the home infrastructure device comprises at least one diagnostic medical device for measuring a value of a fourth patient vital sign. The fourth patient vital sign may be identical with the first, second and/or third vital signs. The method further comprises evaluating the effectiveness of the teleconsultation. The effectiveness is evaluated at least partially by logging the value of the fourth patient vital sign for a second predetermined amount of time. The second predetermined amount of time may or may not be identical with the first predetermined amount of time. Measuring the value of a patient vital sign may be an effective way of measuring how effective a teleconsultation was. For instance in the case of the diabetic tracking the value of the patient's blood sugar level after the teleconsultation may reveal that after the teleconsultation the patient has improved his or her diet and exercise level. As a result the patient's blood sugar level is better under control. This provides an objective way of measuring the effectiveness of the teleconsultation. This has the benefit of being able to provide better healthcare services to the patient for instance if the teleconsultation is determined to have not been effective, then additional consultation by a healthcare professional can be provided. This also provides feedback on which teleconsultations were most effective. The fourth patient vital sign and the first patient vital sign may be different vital signs or they may be the same vital sign. The fourth patient vital sign and the second patient vital sign may be different vital signs or they may be the same vital sign. The fourth patient vital sign and the third patient vital sign may be different vital signs or they may be the same vital sign.

In another embodiment the remote patient management system comprises a healthcare professional user interface. The healthcare professional user interface comprises a microphone. The computer readable storage medium contains a voice recognition module. The method further comprises receiving a command to update images displayed on the patient display using the microphone and the voice recognition module. The method comprises changing the images displayed in the patient display using the command. This embodiment is advantageous because the healthcare professional does not need to manually select when images are changed. This could be implemented in a variety of ways, for instance a specific command to change images could be given or the voice recognition module could be used with a pattern recognition system that determines when the healthcare professional has changed to the next topic automatically. In this case the images are updated according to what the healthcare professional is talking about. The healthcare professional can concentrate on discussing with the patient and not on controlling which images are displayed.

In another embodiment during the step of displaying the images on the patient display during the teleconsultation identical images to those displayed on the patient display are displayed on the healthcare professional display. This embodiment is advantageous because the images displayed on the patient display and the healthcare professional display are identical. In this way the healthcare professional and the patient may have a better conversation about the images shown on the patient display.

In another embodiment the healthcare professional display comprises a healthcare professional user interface. The healthcare professional user interface is adapted for selecting a specific region of an image displayed on the healthcare professional display. The patient display is adapted for indicating the specific region of the image displayed. This embodiment is advantageous because the healthcare professional is able to indicate to the patient a particular area of the image displayed.

In another embodiment the patient display comprises a patient user interface. The patient user interface is adapted for selecting a specific region of an image displayed on the patient display. The healthcare professional display is adapted for indicating the specific region of the image displayed. This embodiment is also advantageous, because the patient may select or indicate a portion of the display and the healthcare professional will know which region of the image the patient is talking about.

In another embodiment the healthcare professional display comprises a healthcare professional user interface. The method further comprises the step of selecting a set of suggested images using the list of topics. The method further comprises displaying the set of suggested images on the healthcare professional display. The step of selecting a set of images using the list of topics is performed by receiving a selection from the healthcare professional user interface. In this embodiment a list of suggested images is displayed on the healthcare professional display. The healthcare professional may look at these images and then select which ones are to be used during the teleconsultation. This allows for convenient and easy customization of the teleconsultation by the healthcare professional.

In another aspect the invention provides for a computer readable storage medium containing instructions that when executed by a computing device of a remote patient management system cause the computing device to perform a method of displaying images on a patient display during a teleconsultation. The computing device comprises a processor for executing the instructions. The remote patient management system comprises a home infrastructure device. The home infrastructure device comprises the patient display. The patient management system comprises a healthcare professional display.

The method comprises accessing a patient record from a patient record database. The method further comprises creating a patient profile using the patient record. The method further comprises identifying a consultation aim using the patient profile. The method further comprises determining a list of topics using the consultation aim. The method further comprises creating a teleconsultation outline using the list of topics. The method further comprises selecting the set of images using the list of topics. The method further comprises displaying the teleconsultation outline on the healthcare professional display during the teleconsultation. The method further comprises displaying the images on the patient display during the teleconsultation.

In another aspect the invention provides for a computer implemented method of displaying images on a patient display during a teleconsultation using a remote patient management system. The remote patient management system comprises a computing device. The computing device comprises a processor for executing the method. The remote patient management system comprises a home infrastructure device. The home infrastructure device comprises a patient display. The patient management system comprises a healthcare professional display.

The method comprises the step of accessing a patient record from a patient record database. The method further comprises the step of creating a patient profile using the patient record. The method further comprises the step of identifying a consultation aim using the patient profile. The method further comprises the step of determining a list of topics using the consultation aim. The method further comprises the step of creating a teleconsultation outline using the list of topics. The method further comprises the step of selecting a set of images using the list of topics. The method further comprises the step of displaying the teleconsultation outline on the healthcare professional display during the teleconsultation. The method further comprises the step of displaying the images on a patient display during the teleconsultation.

### BRIEF DESCRIPTION OF THE DRAWINGS

In the following preferred embodiments of the invention will be described, by way of example only, and with reference to the drawings in which:

FIG. 1 shows a flow diagram which illustrates an embodiment of a method according to the invention;

FIG. 2 shows a flow diagram which illustrates a further embodiment of a method according to the invention;

FIG. 3 shows a block diagram which illustrates a remote patient management system according to an embodiment of the invention;

FIG. 4 shows a block diagram which illustrates a further embodiment of a remote patient management system according to an embodiment of the invention;

FIG. 5 shows a block diagram which illustrates a further embodiment of a remote patient management system according to an embodiment of the invention;

FIG. 6 shows a visualization of the steps performed in an embodiment of the method according to the invention;

FIG. 7 shows an example of assessment parameters that may be used for patient profiling;

FIG. 8 is a diagram which illustrates the relationship between a consultation aim and topics which may be used for creating a teleconsultation outline;

FIG. 9 is a table which shows a relation between the assessment parameters of FIG. 7 in relation to a consultation aim;

FIG. 10 shows rules that may be used for generating a teleconsultation outline;

FIG. 11 shows an example of rules which may be used to select visual content or images;

FIG. 12 shows an example of a user interface or graphical user interface for a healthcare professional according to an embodiment of the invention;

FIG. 13 shows an example of a user interface for a patient according to an embodiment of the invention;

FIG. 14 shows a block diagram which illustrates a further embodiment of a remote patient management system according to an embodiment of the invention; and

FIG. 15 shows a block diagram which illustrates a further embodiment of a remote patient management system according to an embodiment of the invention.

### DETAILED DESCRIPTION OF THE EMBODIMENTS

Like numbered elements in these figures are either equivalent elements or perform the same function. Elements which have been discussed previously will not necessarily be discussed in later figures if the function is equivalent.

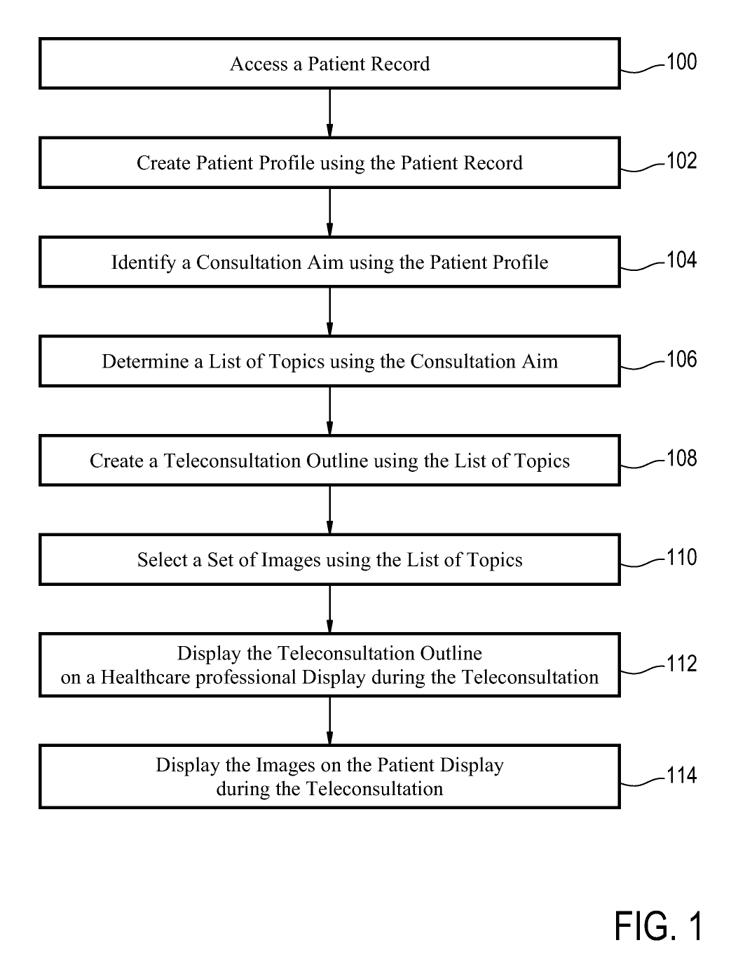

FIG. 1 shows a flow diagram which illustrates an embodiment of a method according to the invention. In step 100 a patient record is accessed. The patient record could be stored in a database, in a collection of files, or locally on the computing device implementing the method. In step 102 a patient profile is created using the patient record. In this step the patient record is scanned or examined for information which is relevant to the patient's current medical condition or which may indicate a medical condition which should be addressed by a healthcare professional. In step 104 a consultation aim is identified using the patient profile. The consultation aim may be a goal or a list of objectives to achieve during a consultation. For instance a patient with a newly diagnosed condition may be identified as needing to provide basic information about the condition. A patient with a chronic condition that the patient is not managing well may need to be provided with information on how to better manage his or her medical condition. In step 106 a list of topics is determined during the consultation aim. In step 108 a teleconsultation outline is created using the list of topics. In step 110 a set of images is selected using the list of topics. In step 112 the teleconsultation outline is displayed on a healthcare professional display during the teleconsultation. In step 114 the images are displayed on the patient display during the teleconsultation.

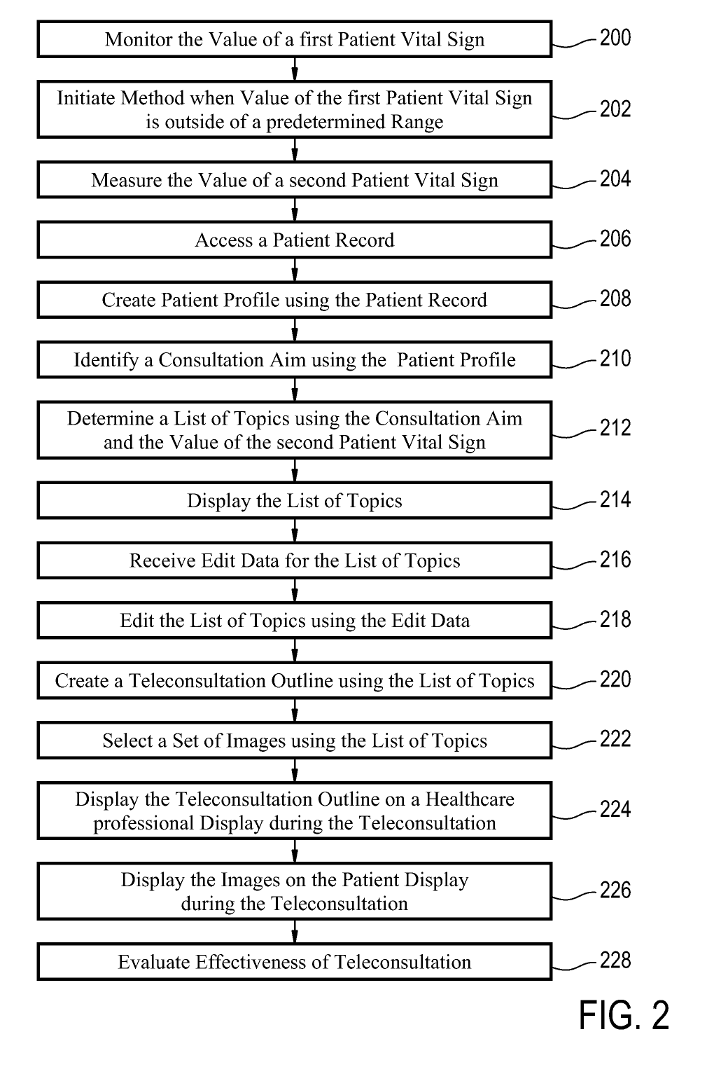

FIG. 2 shows a flow diagram which illustrates a further embodiment of a method according to the invention. In step 200 the value of a first patient vital sign is monitored. In step 202 the method is initiated when the value of the first patient vital sign is outside of a predetermined range. In step 204 the value of a second patient vital sign is measured. The second patient vital sign may be identical or may be different from the first patient vital sign. In step 206 a patient record is accessed. In step 208 a patient profile is created using the patient record. In step 210 a consultation aim is identified using the patient profile.

In step 212 a list of topics is determined using the consultation aim and the value of the second patient vital sign. In step 212 the consultation aim is identified using both the patient's prior medical history via the patient record and also the patient's current medical status by using the second patient vital sign. In step 214 a list of topics is displayed. In step 216 edit data for editing the list of topics is received. This may be achieved for instance by having a graphical user interface which displays the list of topics. The healthcare professional may be able to deselect some topics and to manually add some topics to the list of topics. Once the edit data for the list of topics has been received in step 218 the list of topics is edited using the edit data. In step 220 a teleconsultation outline is created using the list of topics. In step 222 a set of images is selected using the list of topics. In step 214 the teleconsultation outline is displayed on a healthcare professional display during the teleconsultation. In step 226 the images are displayed on the patient display during the teleconsultation. In step 228 the effectiveness of the teleconsultation is evaluated. The effectiveness of the teleconsultation may be evaluated in several different ways. For instance a quiz may be used to evaluate the effectiveness, or the remote patient management system may use a diagnostic medical device to measure one or more patient vital signs. In this case an improvement in the patient's vital sign such as the weight may indicate that the teleconsultation was effective.

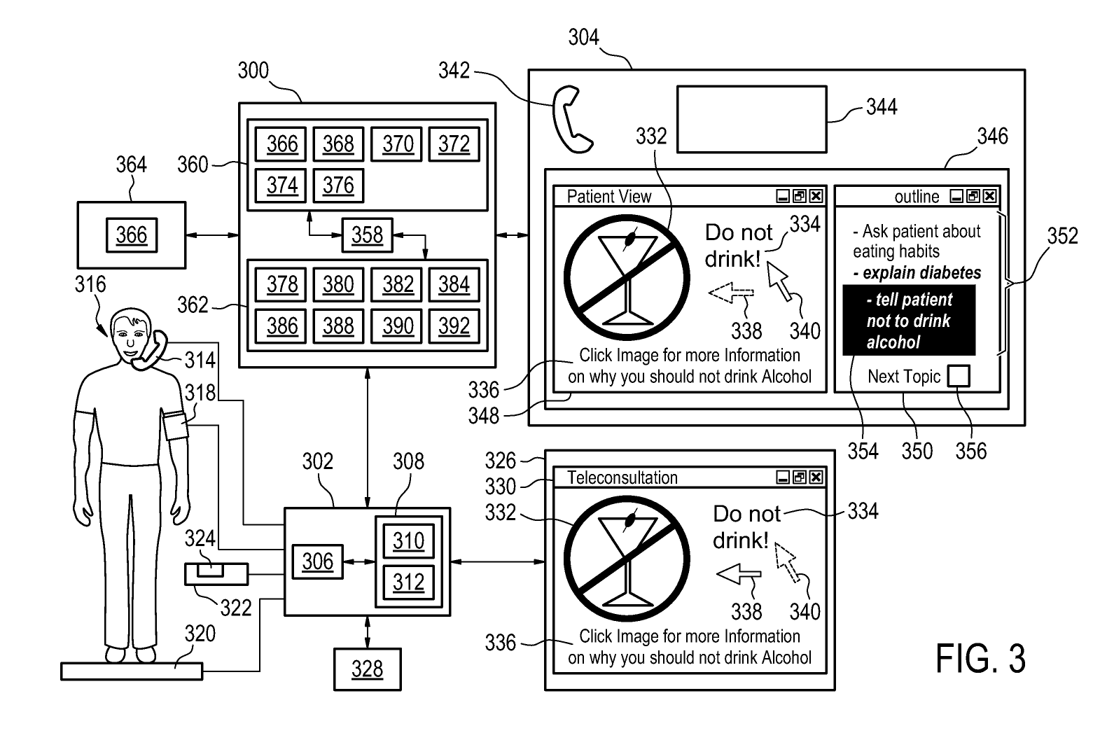

FIG. 3 shows a block diagram which illustrates a remote patient management system according to an embodiment of the invention. The remote patient management system is shown as comprising a computing device 300. The computing device 300 is connected to a home infrastructure device 302 and is also connected to a healthcare professional interface 304.

The home infrastructure device may be connected to the computing device 300 using telephone, networking, internet connection, Ethernet, fiber optic connection, radio communication, or other telecommunication means. The home infrastructure device 302 comprises a processor 306 for executing machine executable instructions. The processor is connected to computer memory 308. The computer memory 308 contains a care plan 310 for execution by the home infrastructure device. The computer memory 308 may also contain a log 312 of a patient vital sign. The computer memory 308 may also contain content elements such as educational content, quizzes and/or surveys. Content elements may be integrated into the care plan 310 or may be delivered on the fly to the home infrastructure device 302 by the computing device 300. The home infrastructure device 302 is shown as being connected to a microphone 314 or telephone. In some embodiments the telephone 314 is separate from the home infrastructure device 302. A patient 316 is able to use the microphone 314 to communicate with a physician during the teleconsultation.

The home infrastructure device is connected to a variety of diagnostic medical devices 318, 322, 320. A blood pressure measurement device 318 such as a blood pressure cuff is shown as being connected to the home infrastructure device. A scale for measuring the weight of the patient 316 is also shown as being connected to the home infrastructure device 302. A blood sugar measurement device 322 is also shown as being connected to the home infrastructure device. The blood sugar measurement device has a receptacle 324 for receiving a blood sample. The home infrastructure device 302 is also shown as being connected to a patient display 326 and a patient user interface 328. The patient display 326 and the patient user interface 328 may both be integrated into the home infrastructure device or may be separate components. In some embodiments the home infrastructure device is a computer in which case the patient display 326 would be the display of the computer and the patient user interface 328 would be the user interface of the computer.

In other embodiments the home infrastructure device 302 is a separate component and the patient display 326 may be a television display. The patient user interface may be integrated into the home infrastructure device 302 in this case or it may be a separate controller. In some embodiments the patient user interface is connected to the home infrastructure device via a wireless or infrared connection.

In an embodiment shown in this Figure the patient display 326 has a graphical user interface window 330. Within the graphical user interface window 330 is displayed an image 332. Adjacent to the image is a motivational message 334. Below the image 332 are optional message 336 or instructions. In this embodiment the patient 316 can use the patient user interface 328 to control the location 338 of a patient cursor. Also shown on the graphical user interface window 330 is the location of the healthcare professional cursor 340. The healthcare professional cursor is the location of a cursor positioned by the healthcare professional. Seeing the location of the healthcare professional cursor 340 on the graphical user interface window 330 allows the patient 316 to see what a healthcare professional is pointing at using the healthcare professional interface 304.

The healthcare professional interface 304 may be a component 300 of the computing device in some embodiments. For instance the computing device 300 could be a laptop or computer, in which case the healthcare professional interface 304 comprises the screen, keyboard, mouse and other components of the computing device 300. In other embodiments the healthcare professional interface is a separate computer or computing device.

If the healthcare professional interface is a separate computer then it would also have a processor and other computer components such as computer memory and computer storage. For the case where the healthcare professional interface is separate from the computing device 300 the computing device 300 and the healthcare professional interface 304 may connect over a variety of telecommunication means. For instance the computing device 300 and the healthcare professional interface 304 may communicate via the internet, via wireless LAN, via a fiber optic connection, via radio, via telephone or other telecommunication means.

The healthcare professional interface 304 is shown as comprising a microphone 342 or telephone. As with the home infrastructure device 302 microphone 342 may be a component of the healthcare professional interface 304 or it may be a separate telephone. The healthcare professional interface 304 is shown as comprising a healthcare professional user interface 344 and a healthcare professional display 346. The healthcare professional user interface 344 comprises a user interface which allows a healthcare professional to interact and control the healthcare professional display and the remote patient management system. The healthcare professional interface may comprise for example a keyboard, mouse, pointer or other computer input device.

The healthcare professional display 346 is shown as displaying two graphical user interface windows 348, 350. The first graphical user interface window 348 shows a graphical user interface that is displaying the same information as the graphical user interface window 330 of the patient display 326. The first graphical user interface window shows the image 332, the motivational message 334 and the instructions 336 in the same way as graphical user interface window 330 did. The position of the healthcare professional cursor 340 is also shown over the first graphical user interface window 348. The location of the patient cursor is also shown 338. The second graphical user interface window 350 shows a teleconsultation outline 352. Several topics of the teleconsultation outline 352 are shown. There is also an emphasized portion 354 of the teleconsultation outline shown. The emphasized portion 354 corresponds to the image 332 which is displayed in graphical user interface windows 330 and 348. Also shown in the second graphical user interface window 350 is a button 356 which allows the healthcare professional to change topics to a different portion of the teleconsultation outline.

Also shown is a patient record database 364 as being connected to the computing device 300. The patient record database 364 may be a component of the computing device 300 or it may be a patient record database in a remote system such as the server of a hospital or physician's office. The patient record database contains a patient record 366 belonging to the patient 316.

Computing device 300 comprises computer storage 360 and computer memory 362. The computer storage 360 has stored within it a patient record 366, a set of images 368, a list of topics 370, predetermined educational content 372, an educational content database 374 and a quiz database 376. The computer memory 362 is shown as containing a remote patient management system control module 378. The remote patient management system control module 378 contains instructions for controlling the operation of the remote patient management system. For instance the remote patient management system control module contains instructions for controlling what is displayed on the patient display 326 and the healthcare professional display 346.

The computer memory 362 also contains a patient profiling module 380. The patient profiling module 380 contains instructions for creating a patient profile using the patient record 366. The computer memory 362 is also shown as containing a consultation aim identification module 382. The consultation aim identification module 382 contains code for generating the consultation aim using the patient profile. The computer memory 362 is also shown as containing a consultation aim identification module 382. The consultation aim identification module contains machine executable instructions for generating a list of topics using the consultation aim. The computer memory 362 is also shown as containing an outline creation module 386. The outline creation module 386 uses the list of topics 370 to generate the teleconsultation outline. This may be achieved using a set of rules or a database. For instance, for a particular topic there may be predetermined outline material which can be integrated into the telecommunications outline. These may be stored in the computer storage 360 or in an external database or data system.

The computer memory 362 is further shown as containing an image selection module. The image selection module uses the list of topics 370 to generate the set of images 368. One way in which the set of images 368 may be generated is that for each item of the list of topics 370 there is an image or group of images that are associated with it. The set of images 368 may be created using images stored locally in the computer storage or may be accessed from a remote location such as a database or data system.

The computer memory 362 is further comprising a voice selection module 390. The voice selection module contains a speech recognition system for receiving voice commands using the microphone 314 for selecting an image 332.

The computer memory 362 is shown as further comprising a teleconsultation evaluation module 392. The teleconsultation evaluation module 392 contains machine executable instructions for evaluating the effectiveness of a teleconsultation. This may be achieved by sending a quiz or survey content element to the home infrastructure device or by reading the log 312 of a patient vital sign.

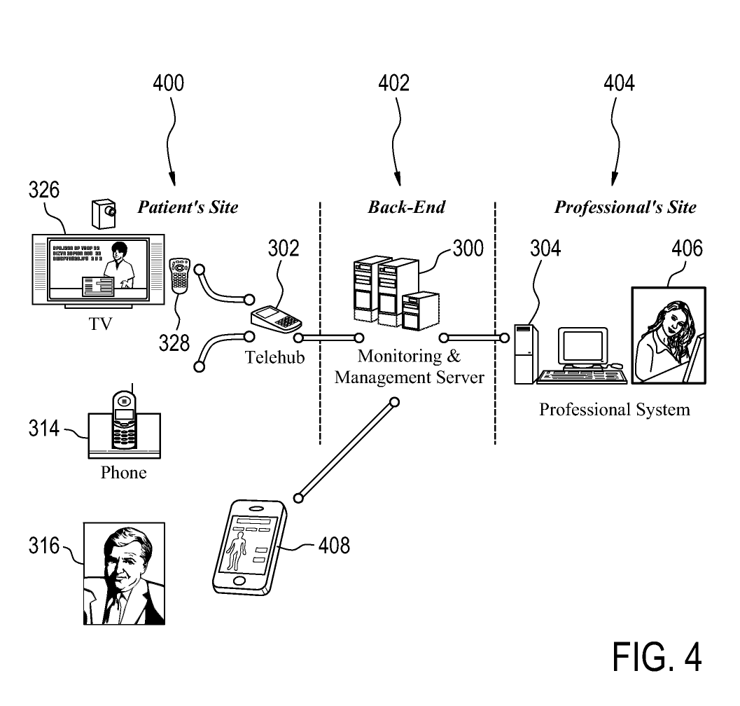

FIG. 4 shows an alternative embodiment of a remote patient management system according to an embodiment of the invention. The remote patient management system shown in FIG. 4 is divided into three physical locations. There is a patient site 400, a back end 402 and the healthcare professional site 404. At the patient site 400 there is located a patient display which is embodied as a television. There is also a patient user interface 328 which is embodied as an infrared controller. The patient 316 communicates using a telephone 314. A telehub embodies the home infrastructure device 302. The telehub 302 is connected to the computing device 300 which is in this embodiment a monitoring and management server. The monitoring and management server 300 is connected to a computer system 304 which is the healthcare professional interface 304 in this embodiment. A healthcare professional 406 uses the healthcare professional interface 304. In an alternative to using a telehub 302 a portable telecommunication device 408 may be used instead. For instance a cellular telephone with a touch-sensitive graphic display may be used instead of the patient display 326.

At the patient site 400, the telehub 302 is the central device. This hub 302 may comprise a processor, communication means, and storage. The telehub 302 may output to a display (e.g. TV, cellular telephone) and telephone (VoIP, cellular telephone). Optionally, there is an interface 328 for the patient to point or highlight specific parts of the visual content. The content highlighted by the patient 316 is in real-time also highlighted at the professional end, to facilitate improved communication.

At the professional site 404 there is a communication means, a processor, a display/User Interface (UI), and storage. The UI of the professional shows the patient profile, visual content, and call script. The main function of this system is to facilitate communication between the different locations by enabling the selection of visual content by the professional 406. The functionality of the professional end is discussed in the next section.

At the back-end 402, there are servers which hold visual data, patient data, and other data related to the use of this system. When a thin client approach is used, the algorithmic functionality can also reside on this location. Preferably, the system is of a web architecture, where both the patient and the professional end are interfaces using a web-browser.

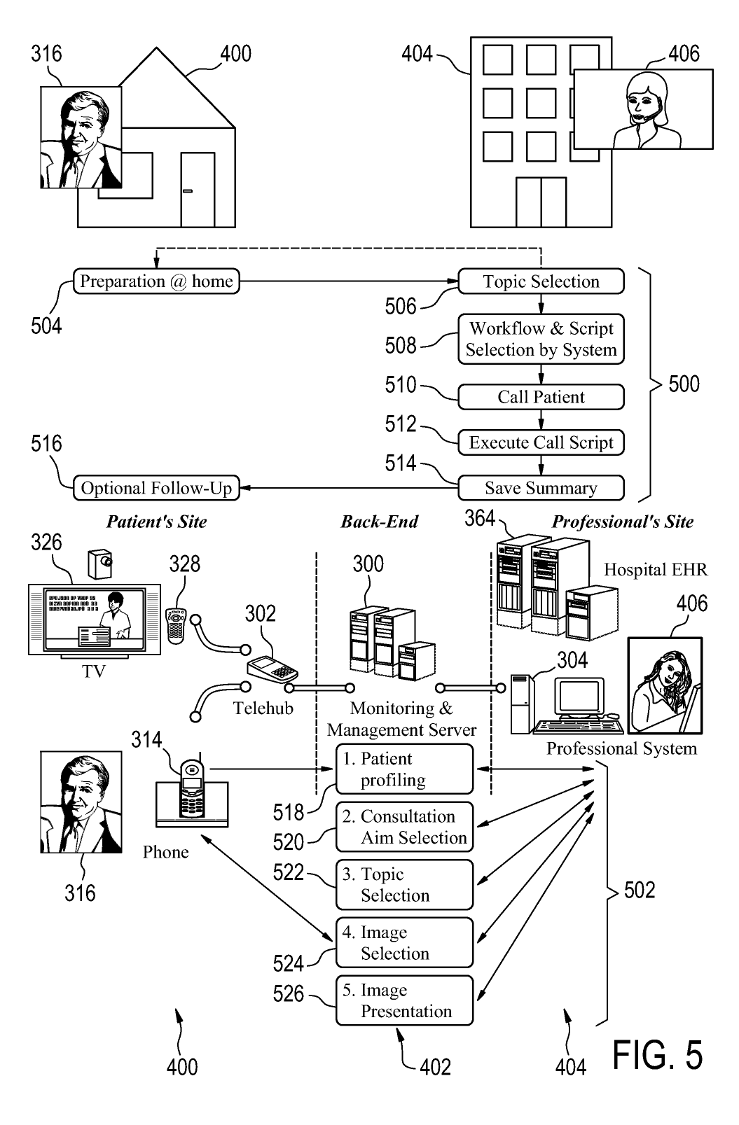

FIG. 5 shows a block diagram illustrating an embodiment of a remote patient monitor according to the invention. FIG. 5 also shows a flow diagram which illustrates the workflow used when operating a remote patient monitor according to an embodiment of the invention. FIG. 5 also shows a flow diagram which illustrates a method 502 according to an embodiment of the invention. As with the embodiment shown in FIG. 4 the embodiment of this remote patient monitor is divided into a patient site 400, a back end 402 and a professional site 404. The embodiment of the remote patient monitor system shown in FIG. 5 is equivalent with that shown in FIG. 4. However, in the embodiment of FIG. 5 a hospital electronic health record 364 located at the professional site 404 is shown. This electronic health record 364 is equivalent with the patient record database 364 shown in FIG. 3.

The workflow 500 illustrates the tasks performed by the patient site 400 and by the healthcare professional site 404. The workflow is shown as starting with either preparation at home 504 or the selection of topics 506. The preparation at home 504 may include study by the patient 316 or the patient 316 be exposed to a care plan. The preparation at home 504 may also include measurements of vital signs by the home infrastructure device 302. In step 506 topic selection is performed. In step 508 workflow and script selection is performed by the system. In step 510 the patient is called by telephone or contacted using the home infrastructure device 302. In step 510 the call script is executed 512. Executing the call script may also include displaying images on the patient display 326. In step 514 a summary is saved in the patient record. 516 is an optional step where follow-up is performed. Follow-up may include sending content elements which review the topics, a quiz to check the effectiveness of the teleconsultation, a survey content element which is used to check the effectiveness of the teleconsultation and/or monitoring the vital signs of the patient 316 using the home infrastructure device 302 for a predetermined amount of time.

The method 502 shown in FIG. 5 has five steps. Step number 1 is patient profiling and is labeled 518. Step number 2 is teleconsultation aim selection. This is the identification of the consultation aim using the patient profile. Step 2 consultation aim selection is labeled 520. Step 3 is topic selection and is labeled 522. Step 4 is image selection and is labeled 524. Step 5 is image presentation and is labeled 526. Steps 1-5 are discussed in greater detail below.

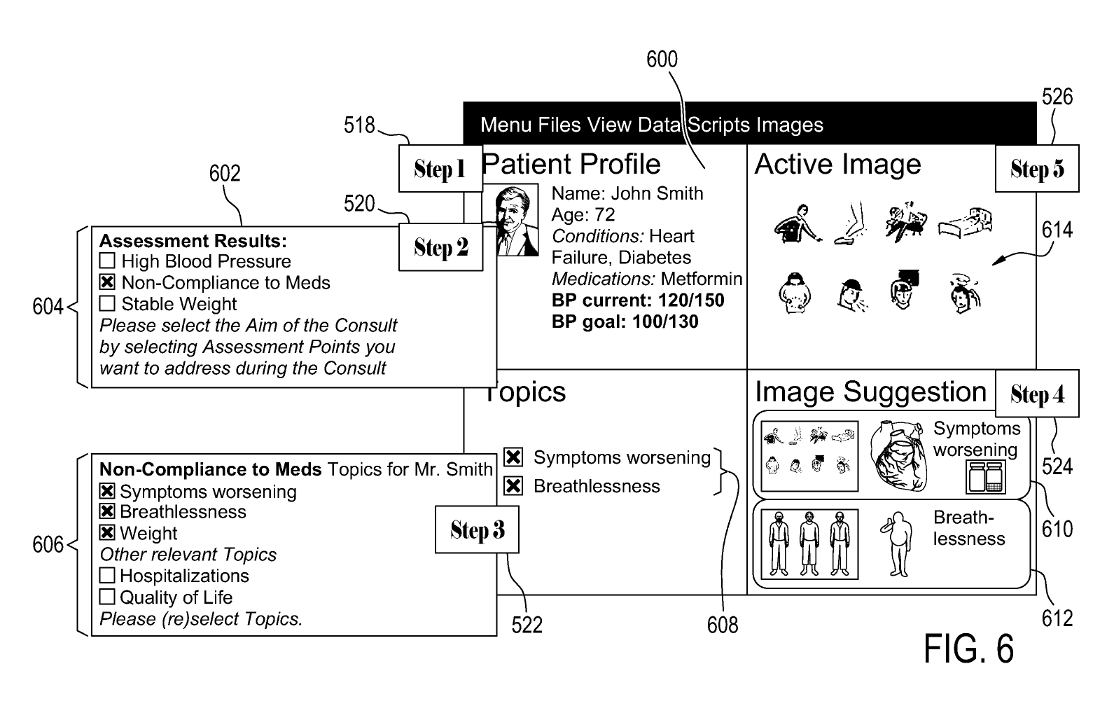

FIG. 6 shows a visualization of the steps performed in an embodiment of the method according to the invention. Steps 1-5 correspond to steps 1-5 518, 520, 522, 524, 526 of FIG. 5. In step 1, 518, a patient profile 600 is created from the patient's patient record. In this example the patient profile has the patient's name, age, medical conditions, medications the patient is taking and also the current blood pressure and blood pressure goal. Using the patient profile 600 in step 2, 520, a consultation aim is selected. Box 602 shows an assessment result created from the patient profile 600. Inside the assessment result 602 are a choice of three consultation aims 604. In this instance the healthcare professional may select 1-3 of these possible consultation aims 604. Alternatively an expert or a rule-based system could be used to automatically suggest or select the consultation aim or aims. After the consultation aim 604 is selected a dialogue box with suggested topics 606 is displayed. 608 shows two topics which were selected for use during the teleconsultation. In step 4, 524, a set of images is selected using the list of topics. Illustrated in step 4 are two sets of images. There is a set of images 610 which correspond to symptoms worsening. The second set of images corresponds to a topic of breathlessness 612. In step 5, 526, the images are presented during the teleconsultation. As an example image 614 is an image which is presented during the teleconsultation.

In step Step 1 [Patient profiling] 518, all the available data from the patient, including his condition, and health status, and possibly also goals for the patient set by the medical professional may be collected. The data used for profiling include current state of the patient based on the answers to the questionnaires as well as the objective patient monitoring data (e.g., vitals). The patient profile 600 is therefore a selection of (relevant) information about the patient that comes from a patient record database (e.g. EHR, patient database, remote patient database). The prompt for the data collection can be triggered directly from the clinical workflow (e.g., based on scheduled consultations from the calendar data of the nurses), or manually by the nurse. In the former case this can be due to a triggered alert, or predetermined number of days in advance of a planned teleconsultation. The predetermined number of days may be specified by a health professional. The patient profile 600 may contain a number of data elements that are indicative of the patient's health-behavior condition.

In step 2 [Select aims of the consultation] 520, the system has collected the data and assesses each element of the profile. A list of assessment results is generated by assessing each element of the profile. This list (cf. 602 in FIG. 6) is a summary of key findings on the patient and serves as the input for the selection of the aim of the consultation. Normally, the aim of the consultation is to address the most prominent problems with the patient, in this example the non-compliance to medications.

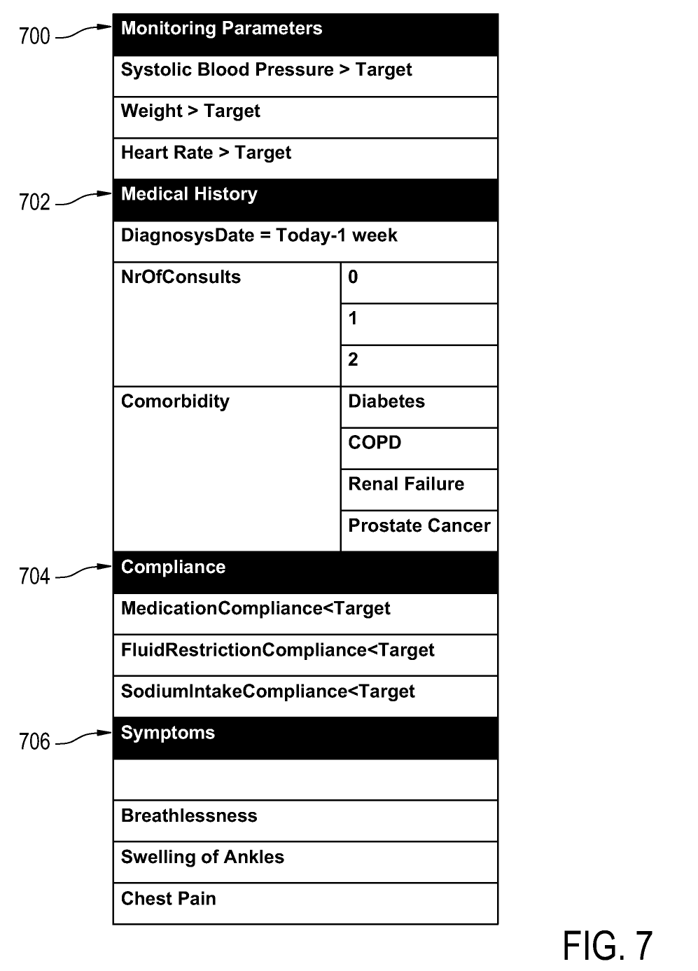

Step 2 can be done automatically using the topic selection algorithm and the results from Step 1. In the table of FIG. 7 examples of parameters and assessment values are given.

FIG. 7 shows an example of assessment parameters that may be used for patient profiling. A patient record may be examined using these assessment parameters to determine and create a patient profile. There are a variety of different assessment parameters illustrated in the table shown in FIG. 7. There are a number of monitoring parameters 700. For the monitoring parameters the systolic blood pressure is checked to see if it is above a particular target. If the weight is above a particular target, or if the heart rate is above a particular target then these monitoring parameters have exceeded an acceptable range. Also shown in FIG. 7 are a number of assessment parameters based on the medical history 702. If the diagnosis date is older than one week, the number of consultations or teleconsultations is examined and co-morbidity factors such as diabetes, COPD, renal failure, or prostate cancer are examined. A number of compliance factors 704 are also examined. A problem would be noted if the medication compliance is less than a particular target that is the patient misses his or her medication more than a specified number of times. If the fluid restriction compliance is below a given target or if the sodium intake compliance is below a given target then a problem is also notated and this is included in the patient profile. A number of symptoms 706 are also listed such as breathlessness, swelling of ankles and chest pain.

In step 3 [Select topics for the consultation], the topics 606 for the consultation are chosen by the system and proposed to the medical professional in a manner shown in FIG. 6. In one embodiment, this step relies on the topic selection algorithm shown in pseudo code below:

```
For every Aim of consultations do
  Make the Aim the root of the topics ontology
  Select a child node of the Aim
  Select a node of the Traverse the ontology tree to mark the For every
    Assessment parameter in the Class do
      Select a node in the domain model that corresponds
        to assessment parameter
Topic from the Table
  End For
  Sort ListOfTopics based on Priority
  Remove duplicates from ListOfTopics
End For
[Optional, professional Input]
Display ListOfTopic on Clinical UI (CUI)
Remove Topics from ListOfTopics that are not highlighted via CUI
```

This algorithm may selects the topics based on, for example, the following classes of input parameters: current symptoms, values of monitoring (vitals), medical history, previous discussed topics, knowledge level, behavioral variables and compliance.

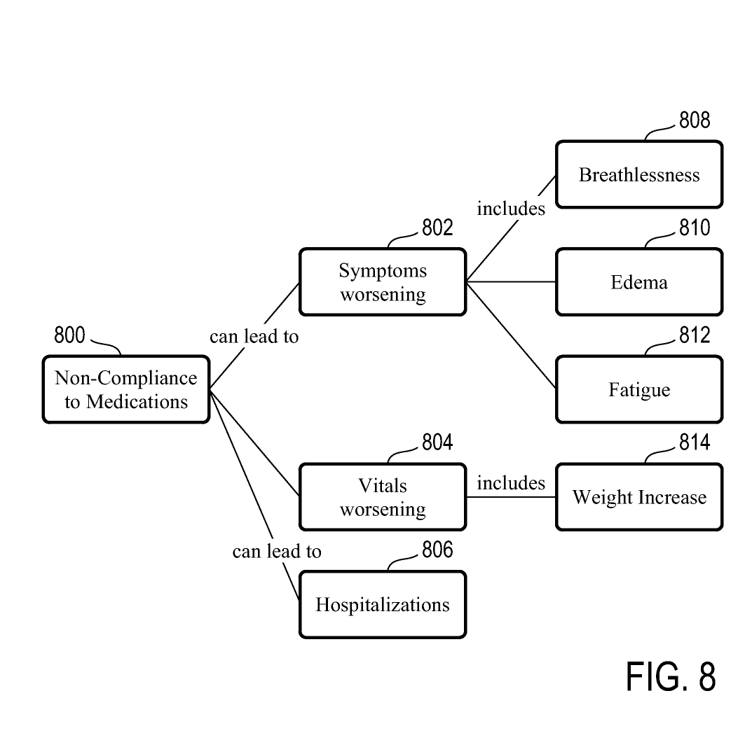

FIG. 8 is a diagram which illustrates the relationship between a consultation aim and topics which may be used for creating a teleconsultation outline. Block 800 illustrates a consultation aim and this is address non-compliance to medications. There is a set of topics that are related to non-compliance to medications, these are: symptoms worsening 802, vitals worsening 804 and hospitalization 806. These general topics also have sub-topics for example symptoms worsening 802 includes breathlessness 808, edema 810 and fatigue 812. Vitals worsening 804 includes weight increase 814.

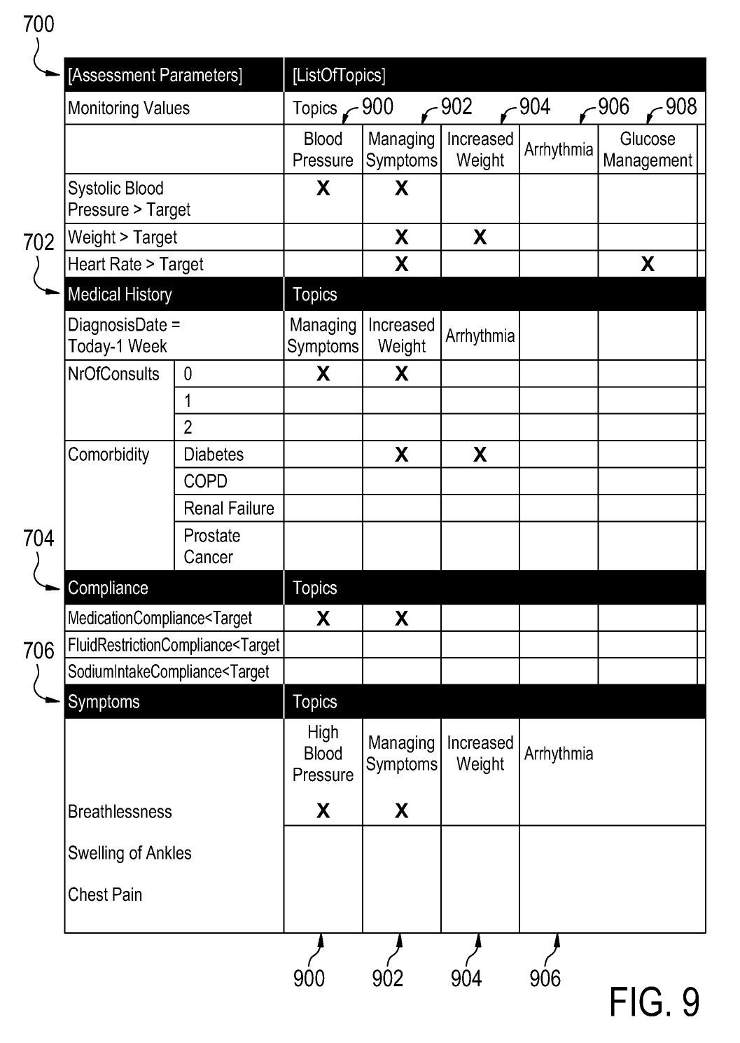

FIG. 9 is a table which shows a relation between the assessment parameters of FIG. 7 in relation to the consultation topic or consultation aim. Each of the assessment parameters 700, 702, 704, 706 shown in FIG. 7 are related to various topics. These include blood pressure 900, managing symptoms 902, increased weight 904, arrhythmia 906, and glucose management 908. For example if the systolic blood pressure is above a target this affects blood pressure 900 and managing symptoms 902. If the weight is above a target value then managing symptoms 902 and increased weight 904 are topics which this affects. If the heart rate is above a given target then this relates to managing symptoms 902 and glucose management 908. If the number of consultations is zero this affects the topics blood pressure 900 and managing symptoms 902. If co-morbidity is diabetes then managing symptoms 902 and increased weight 904 are topics which this affects. If the patient is not taking medication and therefore the medication compliance is below a given target then this may affect blood pressure 900 and managing symptoms 902. If the patient experiences breathlessness then this affects high blood pressure 900 and managing symptoms 902.

In Step 3 [Workflow & script selection] 522 of FIG. 6, the call script is a text-based or visual workflow and comes from the local call centre guidelines or from a remote guideline database, optionally there is a selection box that defines the active script. Additionally, it can be edited with an authoring tool or user interface by the health professional. A script is equivalent to a telecommunication outline.

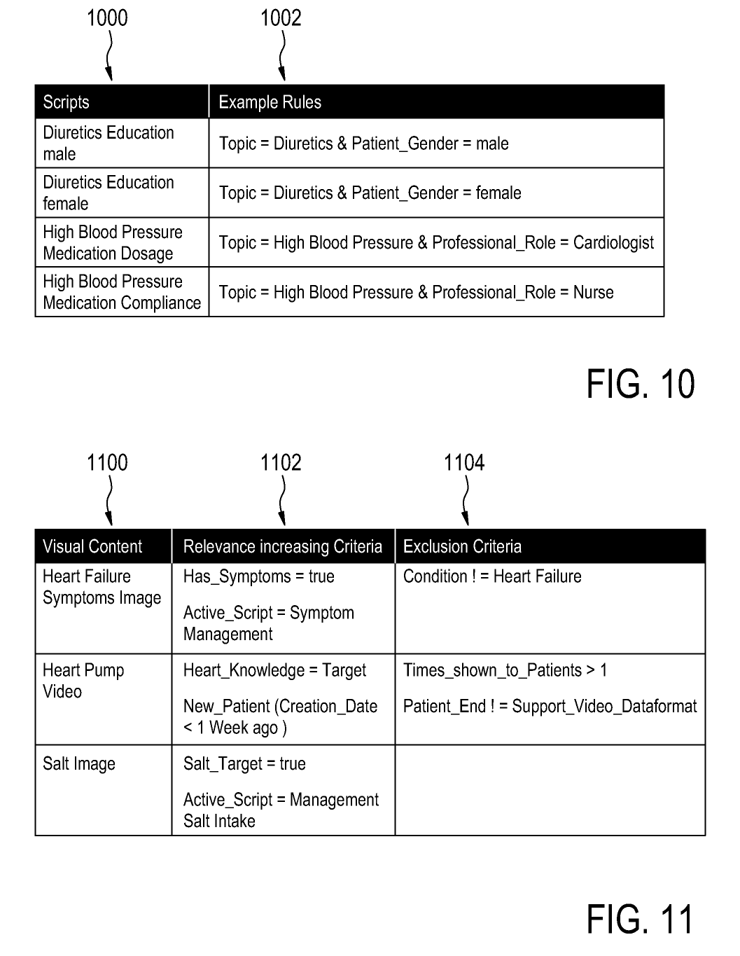

Based on the topic, a workflow and/or script can be selected by the system or manually. The algorithm for selecting the script can take into account personalization for the patient (e.g. by co-morbidities) and the professional (e.g. by role). Examples of algorithmic rules are given in FIG. 10.

FIG. 10 shows rules that may be used for script selection for generating a teleconsultation outline. A teleconsultation outline may also be referred to as a script. Shown in FIG. 10 is a column of scripts 1000 and example rules 1002 which correspond to each of these scripts.

FIG. 11 shows an example of rules which may be used to select visual content or images. There is a column 1100 which shows various visual content or images. Column 1102 lists criteria which indicates that this image is relevant for the patient and should be selected for the set of images. Column 1104 lists exclusion criteria which indicate that a particular element or image 1100 should not be included in the set of images. For example for a heart failure symptoms image it is relevant if the patient has the symptoms of heart failure and is enrolled in a symptom management program. However, if the patient does not have heart failure this image should not be shown to the patient.

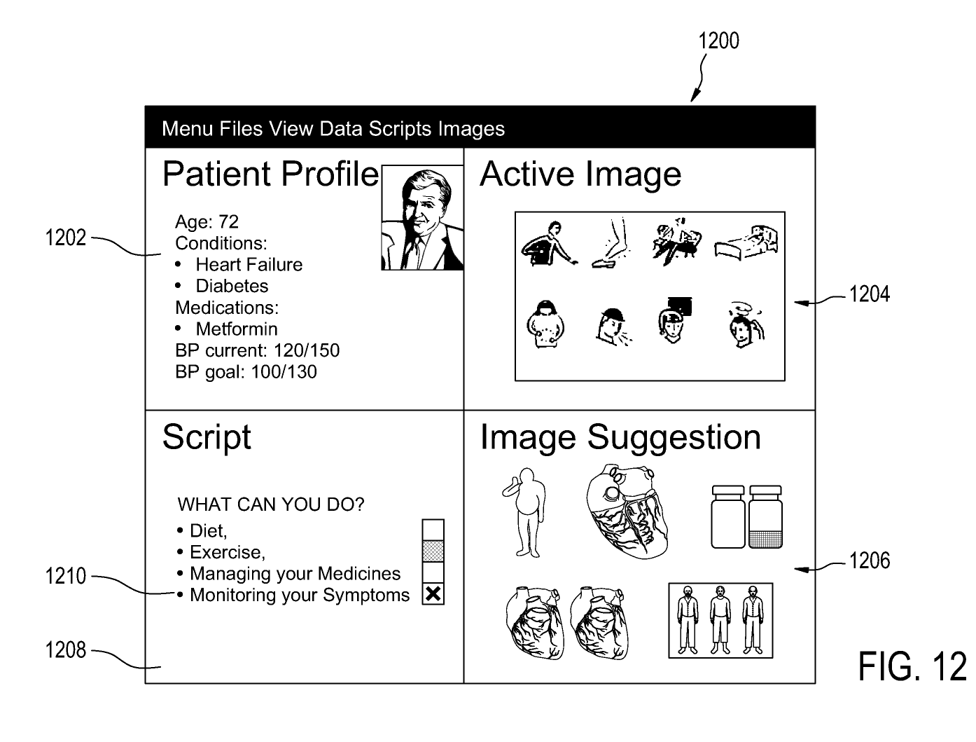

FIG. 12 shows an example of a user interface or graphical user interface for a healthcare professional 1200. The user interface 1200 shows a patient profile 1202 which contains information about the patient. The image which is being actively displayed to the patient 1204 is displayed. This way the healthcare professional knows which image is being presented currently to the patient. There is a region 1206 which contains additional image suggestions or supplementary images that the physician or healthcare professional can choose to show to the patient. There is also a region of the user interface 1200 which shows a script 1208 or teleconsultation outline. A portion of the teleconsultation outline 1210 may be highlighted to indicate what portion of the script 1208 corresponds to the active image 1204.

Upon connection to the patient, the first image is presented to the patient. The health professional goes through the call script and selects each of the images that is presented to the patient.

The suggested visual content panel 1206 is filled with visual content selected by a content selection algorithm. This visual content resides in the storage on one of the three physical locations of the system. The content selection algorithm selects visual content that is relevant to the tele-education based on the patient profile, based on the active call script, and based on the health professional profile (examples of rules are given in FIG. 11). Also, there can be a representation how often this content has been shown to this specific patient, for example by color coding. The information on this statistic comes from, and is saved to, the patient record.

The visual content can be selected online and synchronous or offline and asynchronous. Online means that on selection during a teleconsultation the visual content is sent from the professional end or back-end to the patient's end. Offline means that the content is already available when the trigger to visualize the content is given by the professional. This can be achieved by having all content installed on the patient's end storage, or by sending the visual content automatically at the beginning of, or just before the teleconsultation starts. This sending can be done manually by the professional (the professional makes selects a set of content elements), or automatically (the system sends all content elements based on the active script, or sends a selection of the top relevant content elements. During teleconsultation, when the professional selects visual content to be shown to the patient, the system just sends an event trigger to the telehealth system on the patient's end, which then shows the content on the linked display.

The active visual content 1204 is the content that is shown on the patient's end. Optionally patient or professional can highlight specific aspects of the content. This can be done by communicating the (x,y) position of a pointer on the image/video, and displaying that pointer on the same position on the other end. Optionally have 2 pointers with different color, one for the patient, one for the professional. Another option is to enrich the visual content with a division in elements that are selectable, and highlight in transparency or color. Optionally, in the case of videos, the video can be rewinded or forwarded by both ends, the time stamp is then communicated to the other end.

The call script or teleconsultation outline can be image based, in which a tree of images is shown. Then one of the panels in the professional UI shows text related to the visual content. Another option is to have the script in keywords. Selection of a keyword then triggers the content selection algorithm to provide new images in the relevant images panel. Alternatively, the script shows in color coding behavioral characteristics of the patient on a topic in the call script or in the patient profile panel (e.g. knowledge, compliance).

Optionally there is an algorithm running in the back which writes to a file which scripts have been discussed and what images have been shown, along with their chronological order. Optionally, the audio file of the teleconsultation can be saved. Note that for each of the scripts and images, utilization is saved to the patient record for future use.

Using the summary, an automatic follow-up questionnaire can be sent to the patient. The system could allow a patient to view a replay of the tele-consultation at a later time.

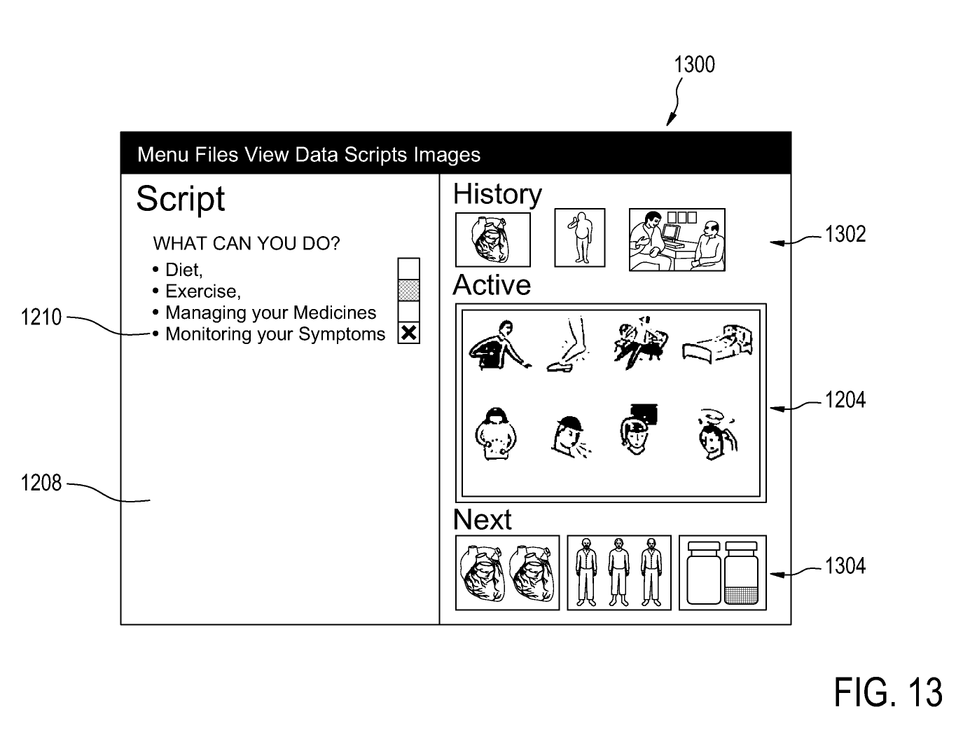

FIG. 13 shows an example of a user interface 1300 for a patient. In this user interface 1300 is shown the script 1208 that was also shown in the healthcare professional's user interface 1200 of FIG. 12. A portion of the script 1210 is also highlighted to indicate the portion of the script that corresponds to the active image 1204. Also shown is a collection of images 1302 which were previously shown to the patient and discussed during the teleconsultation. Additionally there is another set of images 1304 which are then to be shown to the patient during the remainder of the teleconsultation.

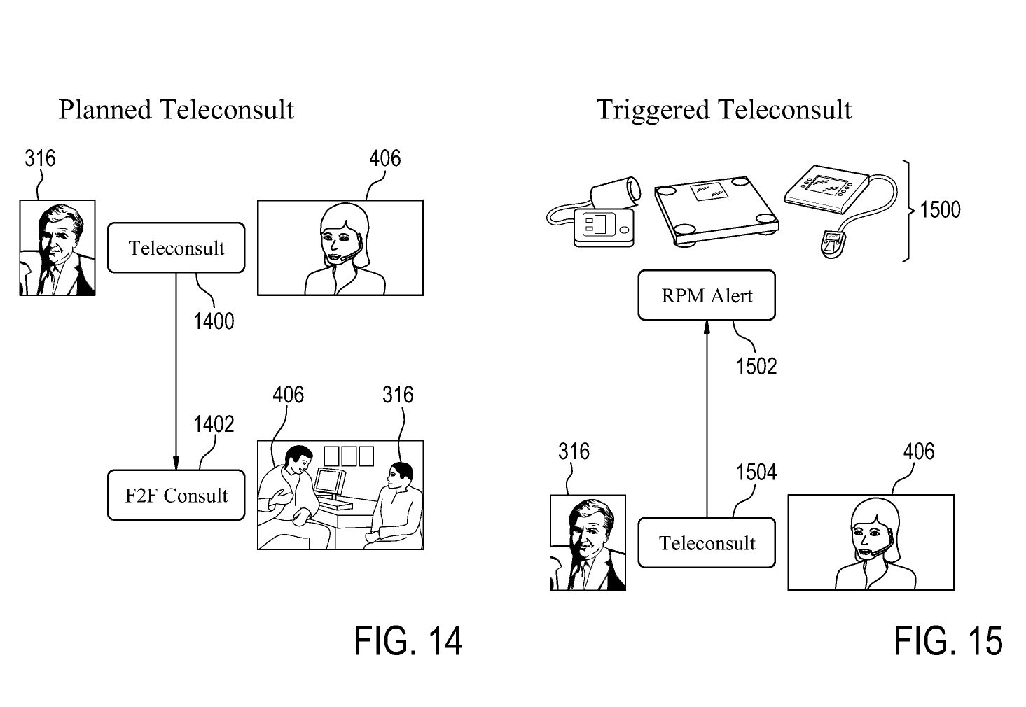

FIG. 14 illustrates how a remote patient management system according to an embodiment of the invention may be used to prepare a patient 316 for a patient face-to-face 1402 consultation with a healthcare provider. In this example the teleconsultation 1400 between the patient 316 and a healthcare provider 406, after the teleconsultation 1400 the patient 316 meets with a healthcare professional 406 in a face-to-face consultation 1402.

FIG. 15 illustrates an embodiment of the invention where a diagnostic medical device 1500 is used by a remote patient management system to generate an alert 1502 that the patient 316 has a medical condition or a change in the patient's medical status which should be addressed by a healthcare professional 406. In this embodiment a remote patient management system alert 1502 triggers a teleconsultation 1504.

While the invention has been illustrated and described in detail in the drawings and foregoing description, such illustration and description are to be considered illustrative or exemplary and not restrictive; the invention is not limited to the disclosed embodiments.

For example, it is possible to operate the invention in an embodiment wherein there is a one-button-click call to the patient. On the call initiation, automatically a connection is made to the patient's station, and the call education UI is displayed on the patient's display. The professional UI shows (or can show) the patient profile, a call script, the active image (with tool base for highlighting) and suggestions for images.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### LIST OF REFERENCE NUMERALS

300 computing device
302 home infrastructure device
304 healthcare professional interface
306 processor
308 computer memory
310 care plan
312 log of patient vital sign
314 microphone or telephone
316 patient
318 blood pressure measurement device
320 scale
322 blood sugar measurement device
324 receptacle for blood sample
326 patient display
328 patient user interface
330 graphical user interface window
332 image
334 motivational message
336 message or instructions
338 location of patient cursor
340 location of health professional cursor
342 microphone or telephone
344 health professional user interface
346 health professional display
348 first graphical user interface window
350 second graphical user interface window
352 teleconsultation outline
354 emphasized portion of teleconsultation outline
356 button
358 processor
360 computer storage
362 computer memory
364 patient record database
366 patient record
368 set of images
370 list of topics
372 predetermined educational content
374 educational content database
376 quiz database
378 remote patient management system control module
380 patient profiling module
382 consultation aim identification module
384 list determination module
386 outline creation module
388 image selection module
390 voice selection module
392 teleconsultation evaluation module
400 patient site
402 back-end
404 healthcare professional site
406 healthcare professional
408 portable telecommunication device
1500 diagnostic medical device

What is claimed is:

1. A remote patient management system comprising:
a computing device comprising a processor;
a home infrastructure device comprising a patient display;
a healthcare professional display; and
a computer readable storage medium, wherein the computer readable storage medium contains instructions that when executed by the processor cause the processor to perform a method of displaying images on the patient display during a teleconsultation, wherein the method comprises the steps of:
accessing a patient record from a patient record database,
creating a patient profile using the patient record,
identifying a consultation aim using the patient profile,
determining a list of topics using the consultation aim,
creating a teleconsultation outline using the list of topics,
selecting a set of images using the list of topics,
displaying the teleconsultation outline on the healthcare professional display during the teleconsultation,
displaying the images on the patient display during the teleconsultation.

2. The remote patient management system of claim 1, wherein the home infrastructure device comprises at least one diagnostic medical device for measuring a value of a first patient vital sign, wherein the method of displaying images on the patient display during a teleconsultation is initiated when the value of the first patient vital sign is outside of a predetermined range.

3. The remote patient management system of claim 1, wherein the home infrastructure device comprises at least one diagnostic medical device for measuring a value of a second patient vital sign, and wherein the determination of the list of topics is at least partially performed using the value of the second patient vital sign.

4. The remote patient management system of claim 1, wherein there is a portion of the teleconsultation outline that corresponds to each image of the set of images, wherein the healthcare professional display is adapted for emphasizing the portion of the teleconsultation outline, and wherein the patient display is adapted for displaying the corresponding image when the portion is emphasized.

5. The remote patient management system of claim 1, wherein the healthcare professional display comprises an healthcare professional user interface, wherein the method further comprises displaying the list of topics on the healthcare professional user interface, wherein the method further comprises receiving edit data for the list of topics, and wherein the method further comprises editing the list of topics using the edit data.

6. The remote patient management system of claim 1, wherein the home infrastructure device comprises at least one diagnostic medical device for measuring a value of a third patient vital sign, wherein method further comprises instructing the home infrastructure device to record a log of the value of the third patient vital sign for a predetermined amount of time, and wherein the determination of the list of topics is at least partially performed using the log.

7. The remote patient management system of claim 1, wherein the patient profile is created by collecting information which indicates a health risk.

8. The remote patient management system of claim 1, wherein the patient display comprises a patient user interface adapted for receiving input from a patient, wherein the patient user interface is adapted for receiving a selection of a displayed image, wherein the home infrastructure device is adapted for sending a signal to the computing device which indicates the selection of the displayed image using the patient user interface, and wherein the method further comprises displaying predetermined educational content on the patient display when the signal is received.

9. The remote patient management system of claim 8, wherein the patient user interface comprises a microphone, wherein the patient user interface is adapted for receiving voice commands using the microphone for receiving the selection of a displayed image.

10. The remote patient management system of claim 1, wherein the method further comprises selecting educational content from an educational content database using the list of topics, wherein the method further comprises delivering the educational content to the home infrastructure device for display on the patent display, and wherein the method further comprises the step of presenting the educational content using the patient display.

11. The remote patient management system of claim 1, wherein the method further comprises constructing a quiz from a quiz database using the list of topics, wherein the method further comprises delivering the quiz to the home infrastructure device for display on the patent display, wherein the method further comprises the step of presenting the quiz using the patient display, wherein the method further comprises the step of scoring the quiz, wherein the method further comprises the step of evaluating the effectiveness of the teleconsultation at least partially using the scored quiz.

12. The remote patient management system of claim 1, wherein the home infrastructure device comprises at least one diagnostic medical device for measuring a value of a third patient vital sign, wherein the method further comprises evaluating the effectiveness of the teleconsultation, wherein the effectiveness is evaluated at least partially by logging the value of the third patient vital sign for a second predetermined amount of time.

13. The remote patient management system of claim 1, wherein the remote patient management system comprises a healthcare professional user interface, wherein the healthcare professional interface comprises a microphone, wherein the computer readable storage medium contains a voice recognition module, wherein the method further comprises receiving a command to update images displayed on the patient display using the microphone and the voice recognition module, and wherein the method comprises changing images displayed on the patient display using the command.

14. The remote patient management system of claim 1, wherein during the step of displaying the images on the patient display during the teleconsultation identical images to those displayed on the patient display are displayed on the healthcare professional display.

15. The remote patient management system of claim 14, wherein the healthcare professional display comprises a healthcare professional user interface, and wherein the healthcare professional user interface is adapted for selecting a specific region of an image displayed on the healthcare professional display, and wherein the patient display is adapted for indicating the specific region of the image displayed.

16. The remote patient management system of claim 14, wherein the patient display comprises a patient user interface, and wherein the patient user interface is adapted for selecting a specific region of an image displayed on the patient display, and wherein the healthcare professional display is adapted for indicating the specific region of the image displayed.

17. The remote patient management system of claim 1, wherein the healthcare professional display comprises a healthcare professional user interface, wherein the method further comprises the step of selecting a set of suggested images using the list of topics; wherein the method further comprises displaying the set of suggested images on the healthcare professional display, and wherein the step of selecting a set of images using the list of topics is performed by receiving a selection from the healthcare professional user interface.

18. A computer readable storage medium containing instructions that when executed by a computing device of a remote patient management system cause the computing device to perform a method of displaying images on a patient display during a teleconsultation; wherein the computing device comprises a processor for executing the instructions; wherein the remote patient management system comprises a home infrastructure device; wherein the home infrastructure device comprises the patient display, wherein the patient management system comprises a healthcare professional display; and wherein the method comprises:
accessing a patient record from a patient record database,
creating a patient profile using the patient record,
identifying a consultation aim using the patient profile,
determining a list of topics using the consultation aim,
creating a teleconsultation outline using the list of topics,
selecting a set of images using the list of topics,
displaying the teleconsultation outline on the healthcare professional display during the teleconsultation,
displaying the images on the patient display during the teleconsultation.

19. A computer implemented method of displaying images on a patient display during a teleconsultation using a remote patient management system; wherein the remote patient management system comprises a computing device; wherein the computing device comprises a processor for executing the method; wherein the remote patient management system comprises a home infrastructure device; wherein the home infrastructure device comprises a patient display; wherein the patient management system comprises a healthcare professional display; and wherein the method comprises:
accessing a patient record from a patient record database,
creating a patient profile using the patient record,
identifying a consultation aim using the patient profile,
determining a list of topics using the consultation aim,
creating a teleconsultation outline using the list of topics,
selecting a set of images using the list of topics,
displaying the teleconsultation outline on the healthcare professional display during the teleconsultation,
displaying the images on the patient display during the teleconsultation.
